(19) United States
(12) Patent Application Publication
(10) Pub. No.: US 2025/0257402 A1
(43) Pub. Date: Aug. 14, 2025

(54) USE OF MMP19 AS BIOMARKER RELATED TO EARLY KIDNEY INJURY IN EARLY DIAGNOSIS OF KIDNEY INJURY

(71) Applicant: RENAL MEDICINE AND BIOTECHNOLOGY CO., LTD., Suzhou (CN)

(72) Inventors: Feng Zheng, Suzhou (CN); Xuejuan Li, Suzhou (CN)

(21) Appl. No.: 18/856,831

(22) PCT Filed: Apr. 10, 2023
(86) PCT No.: PCT/CN2023/087238

§ 371 Date: Oct. 14, 2024

(30) Foreign Application Priority Data
Apr. 15, 2022 (CN) 202210414753.1

(51) Int. Cl. C12Q 1/6883; C12Q 1/6809; G01N 33/573; G01N 33/68

(52) U.S. Cl. CPC C12Q 1/6883; C12Q 1/6809; G01N 33/573; G01N 33/6893; C12Q 2600/118; C12Q 2600/158; G01N 2333/96494; G01N 2800/347; G01N 2800/52

(57) ABSTRACT

The disclosure finds that MMP19 can be used as a biomarker related to early kidney injury and provides the use of a reagent for detecting the biomarker MMP19 in a sample in the preparation of a product for early diagnosis of kidney injury, a product for early diagnosis of kidney injury which comprises the reagent, and a diagnosis method. The product and method can be used to determine whether a subject has kidney injury or is at risk of kidney disease in an early, accurate, rapid and non-invasive manner.

### CROSS-REFERENCE TO RELATED APPLICATIONS

This application is the U.S. National Phase of PCT/CN2023/087238 filed Apr. 10, 2023, which claims priority to Chinese Patent Application No. 202210414753.1, filed Apr. 15, 2022, the entire content of both are incorporated herein by reference in their entirety.

### FIELD OF TECHNOLOGY

The present disclosure belongs to the technical field of biological diagnosis and particularly relates to use of MMP19 as a biomarker related to early kidney injury in the early diagnosis of kidney injury.

### BACKGROUND

There are currently about five million patients with uremia in China, and about 400,000 new cases are added each year. If we conservatively calculate that each patient needs an investment of about 100,000 yuan per year, the current five million patients will require an annual investment of about 500 billion yuan, most of which is the expense for dialysis machines, consumables and medicines imported from abroad.

Early identification and intervention to slow down the development of chronic kidney disease and uremia in people at high risk is the key to solving this major health and economic problem. Early kidney injury includes functional and structural damage to the glomerular filtration membrane and renal tubules, caused by various pathogenic pathways such as various inflammations, metabolic abnormalities and immune injury. People at high risk of chronic kidney disease, such as diabetes, hypertension, and a family history of kidney disease, experience slow and unnoticeable progressive deterioration of kidney lesions.

The detection of early kidney injury indicators plays an important role in the early diagnosis and early intervention of kidney disease. Classic biomarkers such as creatinine and/or urea nitrogen are indicators for monitoring kidney function, but cannot be used as indicators for detecting early kidney injury. Especially in the acute stage, kidney injury is not correlated with changes in these classic indicators.

Therefore, it is urgent to find a flexible, convenient, non-invasive, highly sensitive and specific method for the early detection of kidney injury, so as to achieve early diagnosis of kidney injury.

### SUMMARY

An objective of the present disclosure is to provide use of a reagent for detecting a biomarker in a sample in the preparation of a product for early diagnosis of kidney injury, so as to determine whether a subject has kidney injury or is at risk of kidney disease in an early, accurate, rapid and non-invasive manner.

To solve the above technical problems, the present disclosure adopts the following technical solution:

The present disclosure provides use of a reagent for detecting a biomarker in a sample in the preparation of a product for early diagnosis of kidney injury, wherein the biomarker is MMP19.

Further, the reagent for detecting a biomarker in a sample includes a reagent for detecting the expression level and/or activity of the biomarker's mRNA in the sample, and/or, the reagent for detecting a biomarker in a sample includes a reagent for detecting the expression level and/or activity of the biomarker's protein in the sample.

Further, the reagent is one or more of a primer for specifically amplifying the biomarker, a probe for specifically identifying the biomarker, a conjugate that specifically binds to a protein encoded by the biomarker, or a reagent capable of detecting the activity of the biomarker.

Still further, the conjugate includes one or more of an antibody, a functional antibody fragment or an antibody conjugate that specifically binds to a protein encoded by the biomarker.

Further, the expression level and/or activity of the biomarker MMP19 in the test sample detected with the reagent for the biomarker in the test sample is compared with that in a normal control, and if the expression level and/or activity of the biomarker MMP19 is increased, the subject is determined to have kidney injury or a tendency to suffer from kidney disease or a recurrence of kidney disease or a poor prognosis of kidney disease.

Further, the kidney injury includes acute kidney injury and/or chronic kidney disease.

Further, the test sample is blood and/or urine.

Further, the test sample is a serum sample.

Further, the kidney injury is tubulointerstitial injury.

The present disclosure further provides a product for early diagnosis of kidney injury, the product including a reagent for detecting a biomarker MMP19.

Further, the reagent for detecting the biomarker MMP19 is one or more of a primer for specifically amplifying the biomarker MMP19, a probe for specifically identifying the biomarker MMP19, a conjugate that specifically binds to a protein encoded by the biomarker MMP19, or a reagent capable of detecting the activity of the biomarker MMP19.

Further, the conjugate comprises one or more of an antibody, an antibody functional fragment or an antibody conjugate that specifically binds to a protein encoded by the biomarker MMP19 (matrix metallopeptidase 19).

Further, the product includes a test paper, a reagent strip, a kit, a chip, and a high-throughput sequencing platform.

Further, the kit includes a qPCR kit, an ELISA kit, an immunoblotting kit, a flow cytometry kit, an immunohistochemistry kit, an immunochromatography kit, and an electrogenerated chemiluminescence kit.

The present disclosure further provides an early diagnosis system for kidney injury, including a detection component and a result determining component, wherein the detection component is configured to detect the expression level and/or activity of the biomarker MMP19, and the result determining component is configured to determine, based on the detected expression level and/or activity of the biomarker MMP19, whether the subject has kidney injury or is at risk of kidney disease.

Further, the detection component includes a kit and a detection device matched with the kit, wherein the kit includes the reagent for detecting the biomarker MMP19,
the kit is selected from a qPCR kit, an ELISA kit, an immunoblotting kit, a flow cytometry kit, an immunohistochemistry kit, an immunochromatography kit, and an electrogenerated chemiluminescence kit; and the detection device is selected from a qPCR amplifier, an ELISA device, an immunoblotting device, a flow cytometry device, an immunohistochemistry device, an immunochromatography device, an electrogenerated chemiluminescence device, and an activity test kit device;
the result determining component includes an input module, an analysis module and an output module,
wherein the input module is configured to input the expression level and/or activity of the biomarker MMP19; the analysis module is configured to analyze, based on the expression level and/or activity of the biomarker MMP19, whether the subject has kidney injury or is at risk of kidney disease; the output module is configured to output an analysis result of the analysis module.

The present disclosure further provides a method for early diagnosis of kidney injury.

Specifically, the method includes the following steps:
1) obtaining a test sample from a subject;
2) detecting the expression level and/or activity of a biomarker MMP19 in the test sample from the subject;
3) correlating the detected expression level and/or activity of the biomarker MMP19 with the disease or the risk of disease of the subject; and
4) if the expression level and/or activity of the biomarker MMP19 is increased compared with a normal control, determining that the subject has kidney injury or a tendency to kidney disease or a recurrence of kidney disease or a poor prognosis of kidney disease.

The kit used in the present disclosure may be an existing MMP19 test kit.

The primer for specifically amplifying the biomarker MMP19 and/or the probe for specifically identifying the biomarker MMP19, described in the present disclosure, may be designed based on MMP19 sequence information and prepared by chemical synthesis, and the conjugate that specifically binds to a protein encoded by the biomarker MMP19 may be an antibody of any structure, size, source, and the like or a fragment of the antibody as long as the antibody or the fragment thereof can bind to matrix metallopeptidase 19.

Compared with the prior art, the present disclosure has the following advantages:

The present disclosure discovers for the first time that the MMP19 can be used as a biomarker related to early kidney injury, and also provides the application of the reagent for detecting a biomarker in a sample in the preparation of a product for early diagnosis of kidney injury, so as to determine whether a subject has kidney injury or is at risk of kidney disease in an early, accurate, quick and non-invasive manner, and to perform early intervention of kidney disease.

### BRIEF DESCRIPTION

FIG. 1 shows serum creatinine levels of mice at different time points after acute kidney injury (AKI) modeling in Example 1;

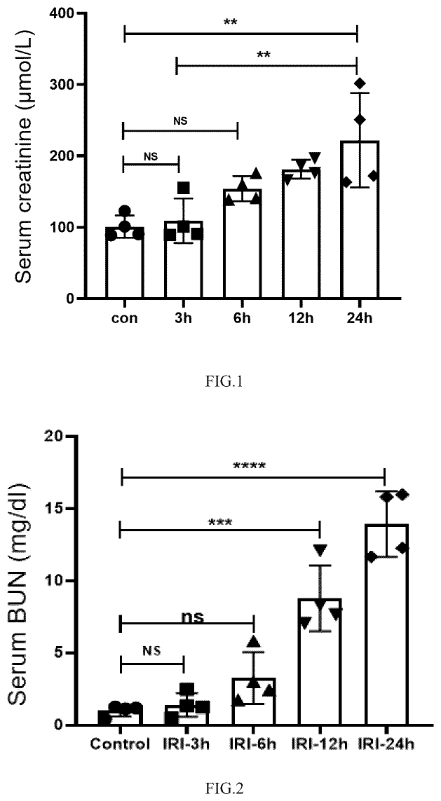

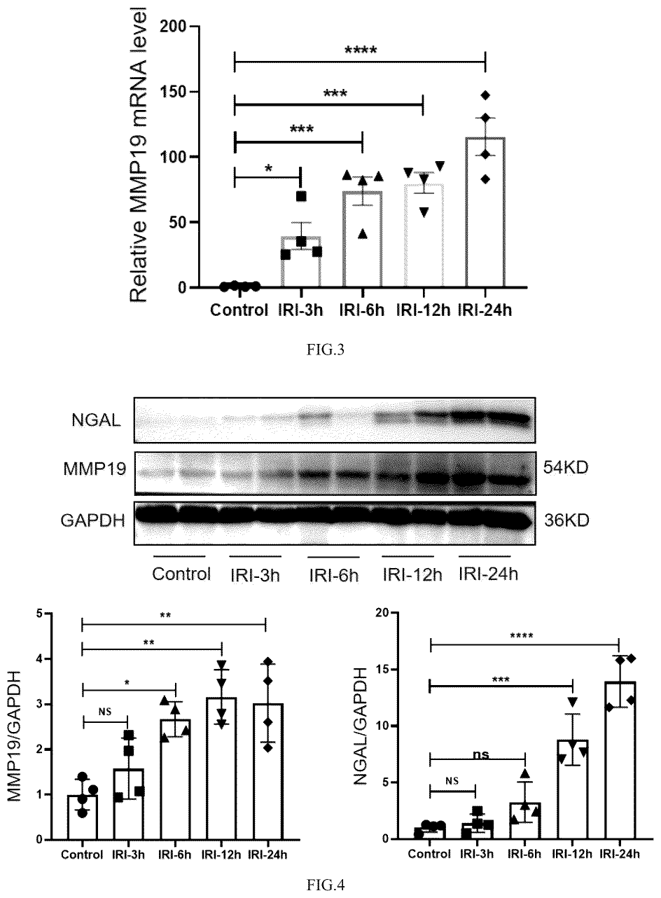

FIG. 2 shows blood urea nitrogen levels of mice at different time points after AKI modeling in Example 1;

FIG. 3 shows mRNA levels of MMP19 in serum of mice at different time points after AKI modeling in Example 1;

FIG. 4 shows experimental results at different time points after AKI modeling in Example 1, where graphs at the top show results of the protein expression levels of MMP19, AKI marker protein NGAL and GAPDH in serum of mice, detected by Western Blot; the left graph at the bottom shows results of comparison of MMP19 content in serum with MMP19 level after correction for GAPDH content; the right graph at the bottom shows results of comparison of NGAL content in serum with NGAL level after correction for GAPDH content;

FIG. 5 shows kidney tissue sections and immunohistochemistry results at different time points after AKI modeling in Example 1, where images at the top show (HE) staining results for pathological analysis of kidney, and images at the bottom show immunohistochemistry results of kidney tissue;

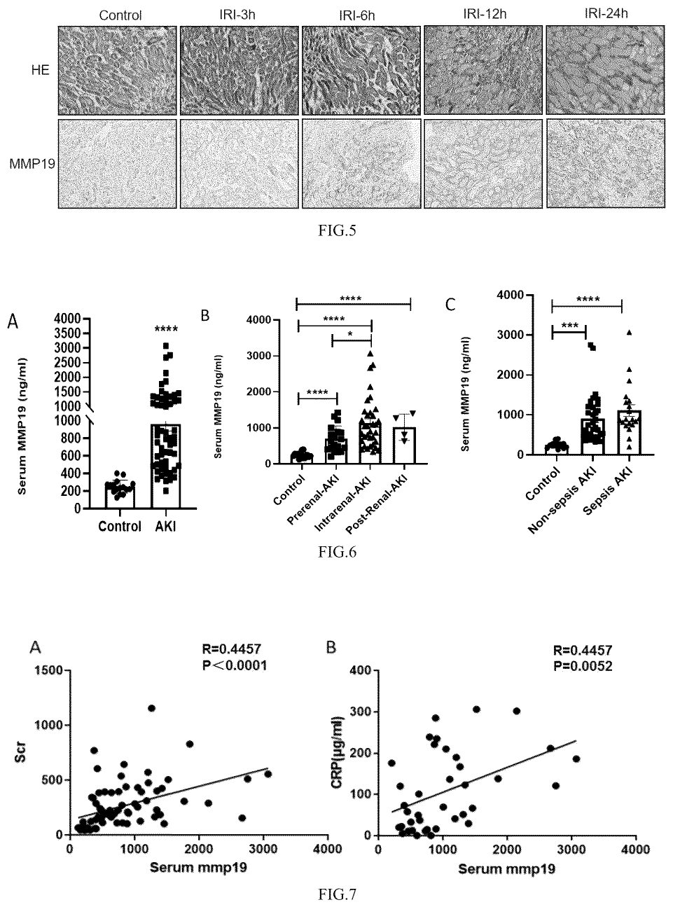

FIG. 6 shows test results of clinical AKI samples in Example 2: A) results of protein expression levels of serum MMP19 of healthy control and AKI samples; B) results of protein expression levels of serum MMP19 of AKI samples classified by different causes of disease; C) results of the protein expression levels of serum MMPP19 of AKI samples classified by whether Sepsis occurs;

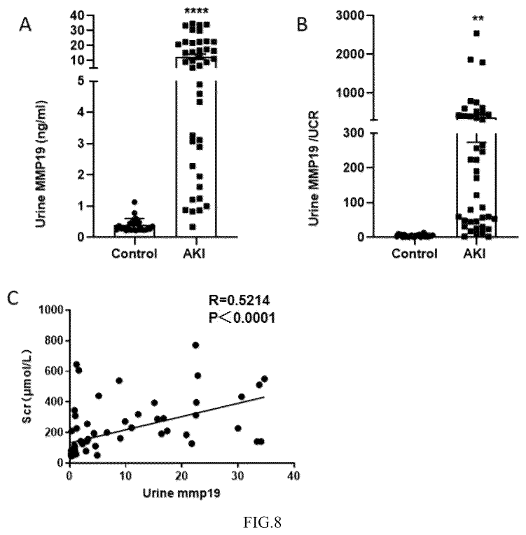

FIG. 7 shows analysis results of correlation between serum MMP19 protein level in clinical AKI samples and clinical indicators in Example 2: A) analysis results of correlation between serum MMP19 protein content and patient's serum creatinine; B) analysis results of correlation between serum MMP19 protein content and patient's C-reactive protein;

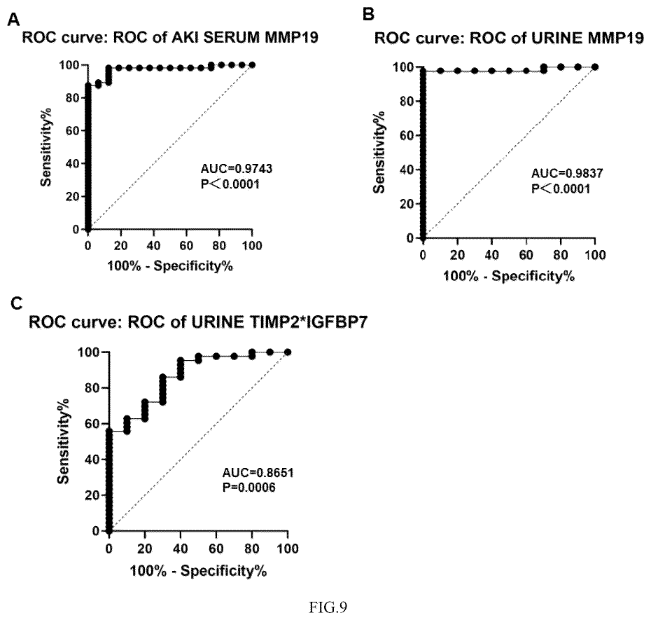

FIG. 8 shows results of protein expression levels of MMP19 in urine samples of AKI patients of different causes: A) results of protein expression levels of urine MMP19 in healthy control and AKI samples; B) comparison results of MMP19 content in urine with MMPP19 level after correction for patient's urine creatinine; C) analysis results of correlation between urine MMPP19 content and serum creatinine;

FIG. 9 shows ROC curves of MMP19 and [TIMP2]*[IGFBP7] for the diagnosis of AKI: A) ROC curve of serum MMP19 protein for the diagnosis of AKI; B) ROC curve of urine MMP19 protein for the diagnosis of AKI; C) ROC curve of urine [TIMP2]*I[IGFBP7](FDA-approved AKI biological markers) for the diagnosis of AKI;

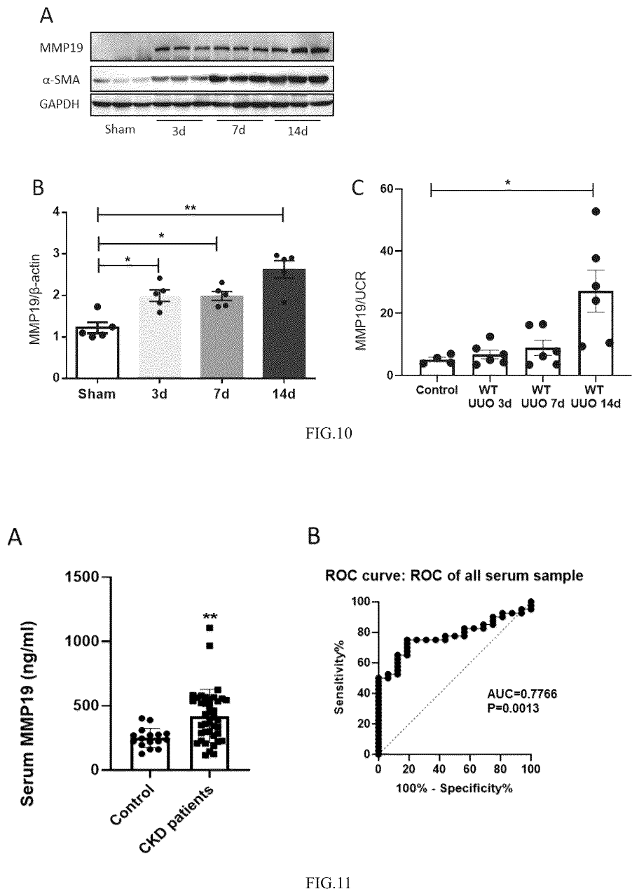

FIG. 10 shows experimental results at different time points after chronic kidney disease (CKD) modeling in Example 3: A) results of protein expression levels of serum MMP19, α-SMA and GAPDH; B) comparison results of MMP19 content in urine with MMP19 level after correction for β-actin; C) comparison results of MMP19 content in urine with MMP19 level after correction for urine creatinine;

FIG. 11 shows protein detection results of MMP19 in clinical CKD samples in Example 4: A) results of serum MMP19 protein levels in healthy control and CKD samples; B) ROC curve of serum MMP19 protein for the diagnosis of CKD;

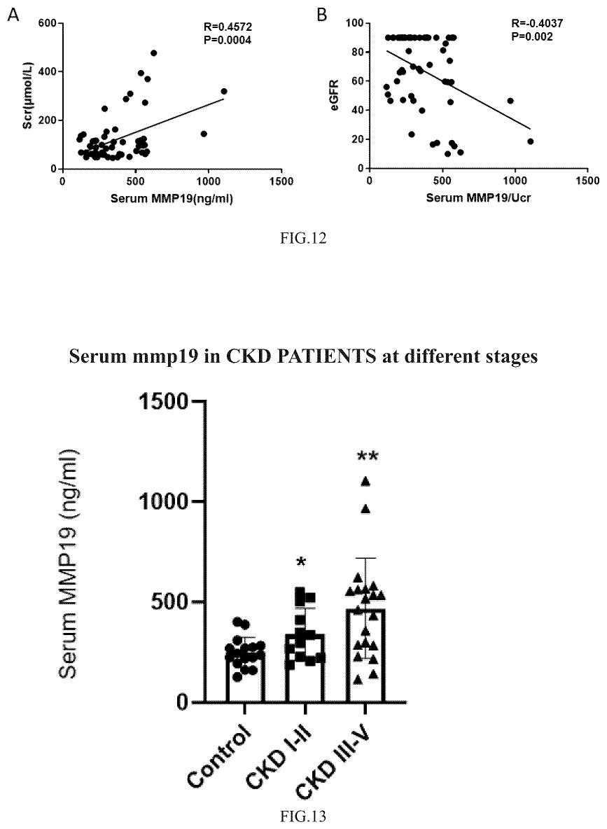

FIG. 12 shows analysis results of correlation between MMP19 protein content in CKD serum samples and clinical indicators in Example 4: A) analysis results of correlation between serum MMP19 protein content and serum creatinine; B) analysis results of correlation between serum MMP19 protein content and glomerular filtration rate;

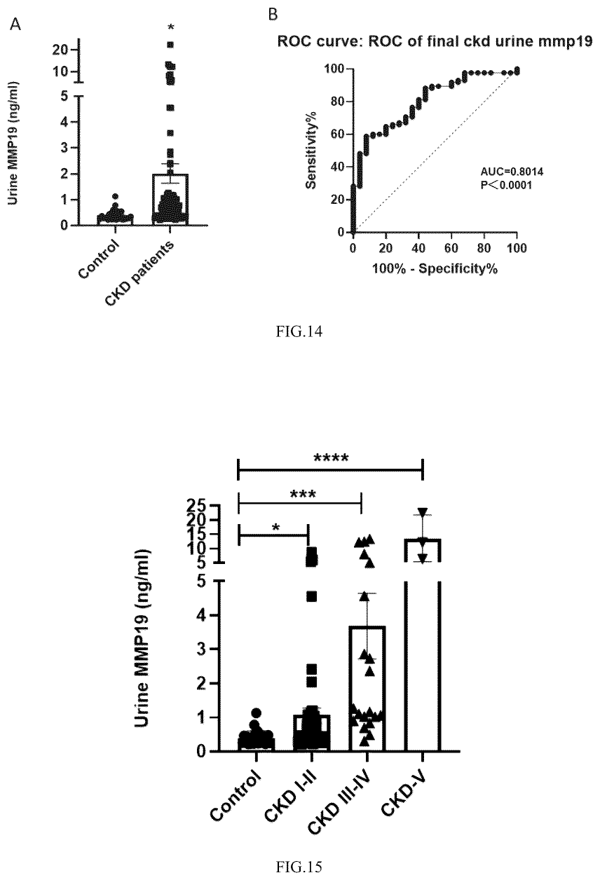

FIG. 13 shows analysis results of MMP19 protein content in clinical serum samples of CKD at different stages in Example 4;

FIG. 14 shows detection results of urine MMP19 in CKD patients in Example 4: A) results of urine MMP19 protein levels in healthy control and CDK samples; B) ROC curve of urine MMP19 protein for the diagnosis of CKD;

FIG. 15 shows analysis results of MMP19 protein content in clinical urine samples of CKD at different stages in Example 4; and

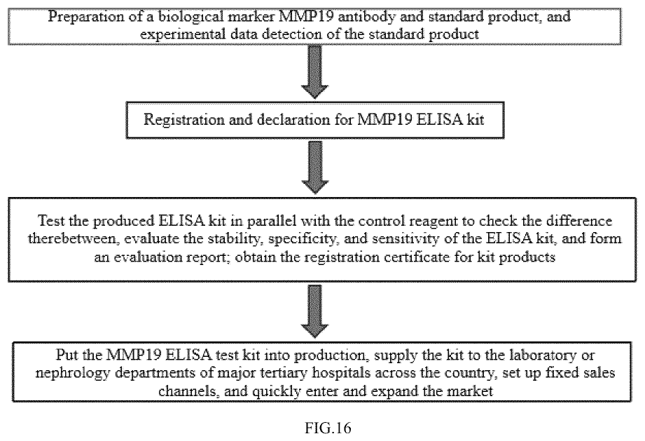

FIG. 16 is a technical route for the development of a kit for early diagnosis of kidney failure.

### DETAILED DESCRIPTION

The disclosure is further described below in conjunction with embodiments. However, the disclosure is not limited to the following embodiments. The implementation conditions used in the embodiments may be further adjusted according to the different requirements of specific use, and the implementation conditions not specified are conventional conditions in this industry. The technical features involved in various embodiments of the disclosure may be combined with each other as long as they do not conflict with each other.

Since creatinine and/or urea nitrogen cannot be used as indicators for detecting early kidney injury and in the acute stage, kidney injury is not correlated with changes in creatinine and/or urea nitrogen indicators. Therefore, the inventors conducted a lot of research and experimental verification and screened out the biomarker MMP19 suitable for early kidney injury.

In the present disclosure, the “biomarker” refers to a compound, preferably a gene. The concentration and/or content of the biomarker in a biological sample from a subject with a first phenotype (e.g., the presence of kidney injury) is different (i.e., increased or decreased) from that in a biological sample from a subject with a second phenotype (e.g., no kidney injury). The concentration and/or content refers to the protein/gene expression concentration or protein/gene content.

In the present disclosure, the concentration and/or content of the biomarker in the above two groups may differ at any level. However, the difference generally exists at the following levels, such as an increase of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, or more. Further, the difference is statistically significant (P<0.05).

In the present disclosure, the “test sample” includes, but is not limited to, serum, plasma, whole blood, blood-derived cells, urine, sweat, kidney tissue and combinations thereof. As a further embodiment, the sample is selected from serum, urine or kidney tissue.

In the present disclosure, the “subject” refers to any animal, including human and non-human animals. Non-human animals include all vertebrates, such as, mammals, such as non-human primates (especially higher primates), sheep, dogs, rodents (such as mice or rats), guinea pigs, goats, pigs, cats, rabbits, cattle, and any livestock or pets; and non-mammals, such as chickens, amphibians, reptiles, etc. In a further embodiment, the subject is a human.

The present disclosure provides a product for early diagnosis of kidney injury. The product includes a reagent for detecting the biomarker of the present disclosure in a sample, and may include instructions for using the kit to determine whether the subject has kidney injury or is at risk of kidney disease.

In the present disclosure, the step of correlating the expression level of the biomarker with a certain possibility or risk can be implemented in different ways. Further, the detected protein concentrations are mathematically combined and related to the underlying diagnosis question.

Further, the logarithmic function used to correlate the biomarker level to the disease preferably employs an algorithm developed and obtained by applying a statistical method. For example, suitable statistical methods are discriminant analysis (DA) (i.e., linear, quadratic, and regular DA), kernel method (i.e., SVM), nonparametric methods (i.e., k-nearest neighbor classifier), PLS (partial least squares), tree-based methods (i.e., logistic regression, CART, random forest method, and boosting/bagging methods), generalized linear models (i.e., logistic regression), principal component-based methods (i.e., SIMCA), generalized additive models, fuzzy logic-based methods, neural network and genetic algorithm-based methods, and correlation analysis.

The area under the curve (AUC) of receiver operator characteristic is an indicator of the performance or accuracy of a diagnosis procedure. The accuracy of a diagnosis method is described by its receiver operator characteristic (ROC). The ROC plot is a line plot of all sensitivity/specificity pairs resulting from continuously varying the decision threshold over the entire range of data observed.

The clinical performance of a laboratory test item depends on its diagnosis accuracy and the diagnosis accuracy refers to the ability of a test item to distinguish a state of the subject in two or more clinical states.

In the specific embodiments of the present disclosure, the raw materials used can be obtained commercially.

In a specific embodiment of the present disclosure, the NCBI accession numbers of MMP19 are NM 002429.6 and NM 001272101.2, the detection of human MMP19 uses an ELISA kit from Biorbyt in the UK: Cat No. orb551482;
the detection of mouse MMP19 uses an ELISA kit from Biorbyt in the UK: Cat No. orb777030;
the MMP19 antibody is purchased from novus: Cat No: NBP2-17311;
the NCBI accession numbers of GAPDH are NM 001289726.1 and NM 008084.3; the detection of GAPDH uses proteintech GAPDH Monoclonal antibody, Cat No. 60004-1-Ig;
the α-SMA antibody is purchased from Cell Signaling Technology, Cat No. 192455;
the NCBI accession number of TIMP2 is NM 003255.5; the detection of TIMP2 uses an ELISA kit from Wuhan CUSABIO, Cat No. CSB-E04733 h;
the NCBI accession numbers of NGAL are NM 001360406.1 and NM 021551.4, and the antibody used for detection is ABCAM, Cat No. ab216462;
the NCBI accession numbers of IGFBP7 are NM 001553.3 and NM 001253835.2, and the detection of IGFBP7 uses an ELISA kit from Wuhan CUSABIO, Cat No. CSB-E17249 h;
serum creatinine and urine creatinine were tested using a creatinine test kit purchased from Nanjing Jiancheng Bioengineering Research Institute, by a sarcosine oxidase method, Cat No. C011-2-1;
blood urea nitrogen (BUN) was tested using test kits purchased from Nanjing Jiancheng Bioengineering Research Institute, using a urease method, Cat No. C013-2-1;
C-reactive protein was tested by immunochromatography;
the eGFR was tested by a simplified MDRD equation modified by the Chinese:

$$eGFR ( m 1 / ( min * 1.73 {m}^{2} ) ) = 186 X ( Scr ) \hat{} - 1.154 X ( age ) \hat{} - 0.203 X ( 0.742 female )$$

Note: eGFR refers to glomerular filtration rate, ml/(min*1.73 m²); Scr is serum creatinine (mg/dl); age is in years; weight is in kg.

### Example 1

### Acute Kidney Injury (AKI) Mouse Experiment

### Renal Ischemia Reperfusion Injury (IRI) Model in Mice

Healthy male wild-type C57BL/6 mice of the same batch, about 8 weeks old, were selected and randomly divided into five groups, with 4 mice in each group. The five groups were a control group (Control or con), a group of mice with the disease model for 3 hours (3 h or IRI-3 h), a group of mice with the disease model for 6 hours (6 h or IRI-6 h), a group of mice with the disease model for 12 hours (12 h or IRI-12 h), and a group of mice with the disease model for 24 hours (24 h or IRI-24 h), respectively. All surgical instruments were sterilized by high pressure, and the abdomen of the mice was depilated in advance.

Preparation of anesthetics: 25 g of tribromoethanol and 15.5 mL of tert-amyl alcohol were mixed to obtain a 1600 mg/mL concentrated tribromoethanol stock solution. After complete dissolution, the stock solution was stored in the dark. The stock solution needs to be diluted into a 20 mg/mL working solution (with saline) before use.

Anesthesia: 20 mg/mL tribromoethanol working solution was injected intraperitoneally at a dose of 350 mg/kg (17.5 μl/g mouse body weight) to anesthetize the mice. The toes of the mice were gently pinched with tweezers to determine whether the mice entered a deep anesthesia state.

Surgery for the model groups: After entering a deep anesthesia, the mice were fixed in a supine position on a 37° C. constant temperature sterile operating table, the abdominal skin was disinfected, and the abdomen was opened. The bilateral kidneys were located with the help of cotton swabs, the fat tissue around the renal pedicles was separated, and after being freed, the renal pedicles were then clamped with vascular clamps for 40 minutes. After the vascular clamps were removed, it was observed with the naked eye that the color of the kidneys returned from purple-black to their original color. After successful reperfusion in kidneys, the incision was sutured and disinfected with iodine, and 500 ul of normal saline was injected intraperitoneally for fluid replacement after the surgery. After waking up, the mice were returned to the cage and given free access to food and water.

Surgery for the control group: During the ischemia reperfusion surgery on the first day, the surgery operations were the same as those for the surgery groups except that the blood vessels were not clamped.

Urine samples were collected, using a mouse metabolic cage, from the mice in the model groups at 3 hours (IRI-3 h), 6 hours (IRI-6 h), 12 hours (IRI-12 h), and 24 hours (IRI-24 h) after modeling, and from the mice in the control group at 24 hours after sham operation and centrifuged at a low temperature (4° C., 3000 rpm, 10 min). After the urine collection, the mouse was killed. The mouse was anesthetized successfully and fixed on a killing board. The abdominal cavity was opened, and blood was collected from the inferior vena cava of the mouse with a 1 ml syringe. The blood collection speed was adjusted according to the filling condition of the inferior vena cava. The needle of the syringe was carefully pulled out, and the blood was injected into an EP tube along the wall of the tube. After standing at room temperature for 1 hour, the blood was centrifuged at 3000 rpm for 10 min, and the supernatant was aspirated and subpackaged. The chest cavity of the mouse was opened, the heart was exposed, the right auricle was cut, and an intravenous infusion needle connected to a 20 ml syringe was inserted from the left ventricle of the mouse. Sterile PBS was perfused until the kidney turned white, the renal capsule was then peeled off, the kidney was removed, and kidney tissue about 3 mm thick in the middle was cut along the short axis of the kidney with a blade, and placed in an EP tube (containing 4% paraformaldehyde) for fixation for more than 24 h, and stored at 4° C. in a refrigerator. Then, kidney tissue about 3 mm thick was cut and then placed in an EP tube filled with an optimal cutting temperature compound (OCT) and the EP tube was then placed in liquid nitrogen. The remaining kidney tissues were subpackaged and placed in liquid nitrogen for subsequent extraction of RNA and tissue protein. The retained urine, serum, kidney tissue protein, and RNA samples of the mouse were all stored at −80° C. in a refrigerator.

At different time points after AKI modeling, the levels of serum creatinine of mice are shown in FIG. 1, and the levels of serum blood urea nitrogen of mice are shown in FIG. 2. Compared with the control group, the model groups had increased serum creatinine and urea nitrogen in mice 12 hours after ischemia reperfusion.

At different time points after AKI modeling, the mRNA levels of MMP19 in serum of mice are shown in FIG. 3. The gene expression of MMP19 in kidney tissue was up-regulated at different time points after AKI modeling. The degree of upregulation increased with time. The mRNA level of MMP19 increased 3 hours after surgery, earlier than the increase in serum creatinine and urea nitrogen of mice.

At different time points after AKI modeling, Western Blot was performed to detect the protein expression levels of MMP19 gene, NGAL gene and GAPDH gene in mouse serum (FIG. 4). The protein expression level of MMP19 gene began to up-regulate 3 hours after modeling, which was significantly earlier than the protein expression level of NGAL gene.

The kidney tissue of mice was subjected to (HE) staining for pathological analysis, and the results are shown in the images at the top of FIG. 5. Immunohistochemistry was performed to detect changes in the protein expression distribution of MMP19, and the results are shown in the images at the bottom of FIG. 5. HE staining showed that histopathological damage in the kidney began to occur 6 hours after modeling, while the immunohistochemistry results of MMP19 showed that the expression of MMP19 was significantly up-regulated 3 hours after modeling, earlier than the pathological damage in the kidney.

### Example 2

### Clinical AKI Sample Detection Experiment

Collection of serum and urine samples from AKI patients: Morning urine/random urine was collected. The urine sample was centrifuged at a low temperature (4° C., 3000 rpm, 10 min). The urine supernatant was taken and subpackaged at 1 mL/tube and stored at −80° C. in a refrigerator. In addition, a tube of serum sample from each patient undergoing renal puncture biopsy was collected on the day of biopsy, where the vacuum blood collection tube with biochemical serum coagulant was used for specimen collection. Immediately after blood collection, the sample was mixed homogeneously by overturning the tube up and down for 8 times, and then allowed to stand for 15-20 min, and then centrifuged at a low temperature (4° C., 3000 rpm, 10 min). The supernatant was then taken and subpackaged at 20 μl/tube, and then stored at −80° C. in a refrigerator.

Samples were collected from patients with clinical diagnosis of acute kidney injury. The patients selected should meet the following conditions: 1. Within 48 hours, the average of serum creatinine indicator is greater than 26.4 μmol/L. 2. Within one week, the serum creatinine level increases to more than 1.5 times the baseline level (the basic value of the patient himself). 3. Urine volume is less than 0.5 ml/h for 3-4 consecutive days. Patients will be selected in the group as long as their test results meet any one of these conditions. Blood and urine samples were collected from the patients in the group.

Collection of samples from the healthy control group: Samples were collected from patients undergoing physical examinations at the physical examination center. The patients selected should meet the following conditions: their age and sex should match those of the patients undergoing renal puncture biopsy and patients with acute kidney injury, and they should have normal renal function test results. The samples, collected in the laboratory after testing, and the steps of sample collection, processing and storage were the same as above.

In this example, we collected 16 serum samples from healthy patients having physical examination as the healthy control group and 56 serum samples from AKI samples.

The serum MMP19 levels of the healthy control group and acute kidney injury samples are shown in FIG. 6A. Compared with the healthy control group, the serum MMP19 levels in patients in the AKI disease group were significantly up-regulated. The average of serum MMP19 level in the control group was 248.9±18.91 ng/ml, and the average of serum MMP19 level in AKI patients was 966.4±83.14 ng/ml.

Patients in the AKI disease group were classified according to different causes of the disease. The serum MMP19 levels of AKI samples classified by different causes of the disease are shown in FIG. 6B. There were 19 patients with Prerenal-AKI, with an average serum MMP19 level of 695±81.49 ng/ml; 34 patients with Intrarenal-AKI, with an average serum MMP19 level of 1130+120.8 ng/ml; and 4 patients with Post-Renal-AKI, with an average serum MMP19 level of 1023±183.4 ng/ml. One of the cases has both Prerenal-AKI and Intrarenal-AKI.

The AKI patients were divided into Sepsis AKI and Non-sepsis AKI according to whether they had Sepsis at the same time. The serum MMP19 levels are shown in FIG. 6C. There were 36 cases of Non-sepsis AKI with an average serum MMP19 level of 913.6±95.34 ng/mL and 20 cases of Sepsis AKI with an average serum MMP19 level of 1091±186.2 ng/mL.

The prism 8.0 correlation was used to analyze the correlation between the serum MMP19 content and the patient's serum creatinine, urea nitrogen, CRP, etc. The results are shown in FIG. 7. The serum MMP19 content was positively correlated with the patient's serum creatinine (FIG. 7A), and the serum MMP19 content was positively correlated with the patient's C-reactive protein (CRP) (FIG. 7B). The content of MMP19 in serum also showed a correlation with blood urea nitrogen, but there was no statistical difference.

In addition, urine samples were collected from 43 AKI patients of different causes and from 25 age- and sex-matched controls and then tested for the content of MMP19. The results are shown in FIG. 8.

Referring to FIG. 8A, compared with the healthy control group, the secretion of MMP19 in the urine of AKI patients in the AKI disease group increased significantly; specifically, the average of urine MMP19 content in the control group was 0.3896±0.04234 ng/ml; the average of urine MMP19 content of AKI patients was 12.44±1.708 ng/ml. FIG. 8B shows adjusted MMP19 levels based on the patients' urine creatinine levels, where the average of MMP19 level in the healthy control group was 4.648±0.6982, while the average of urine MMP19 content of AKI patients was 346.8±80.91. The prism 8.0 correlation was used to analyze the correlation between the urine MMP19 content and the patient's clinical indicators (FIG. 8C). The urine MMP19 content was positively correlated with the patient's serum creatinine.

The area under the ROC curve (AUC) was used to evaluate the sensitivity and specificity of MMP19 as a diagnostic marker and compared with urine [TIMP2]*[IGFBP7](FDA-approved AKI biomarkers). The results are shown in FIG. 9. The results show that the area under the ROC curve for serum MMP19 in the diagnosis of AKI is 0.9743 (FIG. 9A), and the area under the ROC curve for urine MMP19 in the diagnosis of AKI is 0.9837 (FIG. 9B), while the area under the ROC curve for the FDA-approved AKI biomarkers urine [TIMP2]*[IGFBP7](TIMP2 content multiplied by IGFBP7 content) in the diagnosis of AKI is 0.8651 (FIG. 9C, using the collected urine samples from 43 AKI patients and 10 age- and sex-matched control samples). Urine MMP19 showed a higher efficacy in the diagnosis of AKI. Therefore, MMP19 is suitable as an effective biomarker for the early diagnosis of acute kidney injury.

### Example 3

### Chronic Kidney Disease (CKD) Mouse Experiment

### Unilateral Ureteral Obstruction (UUO) Model

Healthy male wild-type C57BL/6 mice of the same batch, about 8 weeks old, were selected and randomly divided into four groups, which were a control group (Sham or Control), a group of mice with the disease model for 3 days (3 d or UUO 3 d), a group of mice with the disease model for 7 days (7 d or UUO 7 d), and a group of mice with the disease model for 14 days (14 d or UUO 14 d). There were 4 mice in the control group and 6 mice in each model group. All surgical instruments were sterilized by high pressure, and the abdomen of the mice was depilated in advance.

Preparation of anesthetics: 25 g of tribromoethanol and 15.5 mL of tert-amyl alcohol were mixed to obtain a 1600 mg/mL concentrated tribromoethanol stock solution. After complete dissolution, the stock solution was stored in the dark. The stock solution needs to be diluted into a 20 mg/mL working solution (with saline) before use.

Anesthesia: 20 mg/mL tribromoethanol working solution was injected intraperitoneally at a dose of 350 mg/kg (17.5 μl/g mouse body weight) to anesthetize the mice. The toes of the mice were gently pinched with tweezers to determine whether the mice entered a deep anesthesia state.

Surgery for the model groups: The anesthetized mice were fixed in a supine position on a sterile operating table, and the abdominal skin was disinfected with 75% alcohol. A 1 cm longitudinal incision was made 1 cm above the midpoint of groin on the left lower abdomen of the mouse, and the abdominal cavity was opened. Firstly, the left kidney was located with the help of a sterile cotton swab; then, the position of the left ureter close to the kidney was determined to separate the surrounding fat tissue, and the left ureter was ligated with thread at the 1/3 position. To ensure the successful construction of the model, the left ureter was ligated again at the position about 1 cm below the ligation point. After ligation, the intestinal tract was repositioned, the abdominal cavity was sutured, the incision was disinfected with iodine, and the mouse was returned to the cage after waking up.

Surgery for the control group: Operations were the same as those for the surgery group except that the ureter was not ligated.

Urine samples were collected, using a mouse metabolic cage, from the mice in the model groups at 3 days, 7 days and 14 days after modeling, and from the mice in the control group at 14 days after sham operation and centrifuged at a low temperature (4° C., 3000 rpm, 10 min). After the urine collection, the mouse was killed. The mouse was anesthetized successfully and fixed on a killing board. The abdominal cavity was opened, and blood was collected from the inferior vena cava of the mouse with a 1 ml syringe. The blood collection speed was adjusted according to the filling condition of the inferior vena cava. The needle of the syringe was carefully pulled out, and the blood was injected into an EP tube along the wall of the tube. After standing at room temperature for 1 hour, the blood was centrifuged at 3000 rpm for 10 min, and the supernatant was aspirated and subpackaged. The chest cavity of the mouse was opened, the heart was exposed, the right auricle was cut, and an intravenous infusion needle connected to a 20 ml syringe was inserted from the left ventricle of the mouse. Sterile PBS was perfused until the kidney turned white, the renal capsule was then peeled off, the kidney was removed, and kidney tissue about 3 mm thick in the middle was cut along the short axis of the kidney with a blade, and placed in an EP tube (containing 4% paraformaldehyde) for fixation for more than 24 h, and stored at 4° C. in a refrigerator. Then, kidney tissue about 3 mm thick was cut and then placed in an EP tube filled with an optimal cutting temperature compound (OCT) and the EP tube was then placed in liquid nitrogen. The remaining kidney tissues were subpackaged and placed in liquid nitrogen for subsequent extraction of RNA and tissue protein. The retained urine, serum, kidney tissue protein, and RNA samples of the mouse were all stored at −80° C. in a refrigerator.

Western Blot was performed to detect the protein levels of serum MMP19 gene, renal fibrosis-related marker protein α-smooth muscle actin (α-SMA) gene and GAPDH gene. FIG. 10A shows that compared with the control group (Sham), the gene and protein levels of MMP19 in mouse kidney tissue were significantly up-regulated in the model group, and the up-regulation level increased with ureteral obstruction time. The protein expression level of MMP19 gene began to up-regulate 3 days after modeling, which was significantly earlier than the protein expression level of α-SMA gene.

The ELISA was performed to detect the MMP19 content in mouse urine. FIG. 10B shows the comparison of the MMP19 content in urine with the MMP19 level after correction for β-actin. FIG. 10C shows the comparison of the MMP19 content in urine with the MMP19 level after correction for urine creatinine. The results showed that the secretion of MMP19 increased in the UUO models, and increased significantly with the obstruction time. Due to the unilateral ureteral ligation in the experiment, the renal function of the mice was normal, and the serum creatinine and blood urea of the mice were normal. However, the MMP19 level in the urine of the mice began to increase on the 3rd day of the UUO mouse model and showed significant levels on the 14th day with statistical significance, indicating that MMP19 can be used as an effective biomarker for the early diagnosis of fibrosis in chronic kidney disease.

### Example 4

### Clinical CKD Sample Detection Experiment

Serum and urine samples from patients undergoing renal puncture biopsy in the Department of Nephrology were collected on the day of biopsy. Morning urine from patients undergoing renal puncture biopsy was collected on the day of biopsy and centrifuged at a low temperature (4° C., 3000 rpm, 10 min). The urine supernatant was taken and subpackaged at 1 ml/tube and stored at −80° C. in a refrigerator. In addition, a tube of serum sample from each patient undergoing renal puncture biopsy was collected on the day of biopsy, where the vacuum blood collection tube with biochemical serum coagulant was used for specimen collection. Immediately after blood collection, the sample was mixed homogeneously by overturning the tube up and down for 8 times, and then allowed to stand for 15-20 min, and then centrifuged at a low temperature (4° C., 3000 rpm, 10 min). The supernatant was then taken and subpackaged at 20 μl/tube, and then stored at −80° C. in a refrigerator.

In this example, 16 serum samples from healthy patients having physical examination were collected as the control group, and 40 samples of patients with renal puncture biopsy were collected.

The serum MMP19 levels of the healthy control group and CKD samples are shown in FIG. 11A. When the CKD group was compared with the healthy control group, the average of MMP19 level in the serum of the healthy control group was 248.9±18.91 ng/ml; the average of MMP19 content in the serum of all renal puncture biopsy samples was 420.2±32.64 ng/ml, and the area under the curve of serum MMP19 for the diagnosis of CKD was 0.7766 (FIG. 11B).

The prism 8.0 correlation was used to analyze the correlation between the MMP19 content in all serum samples and the corresponding serum creatinine. FIG. 12A shows that the MMP19 content in serum is positively correlated with serum creatinine, R=0.4572, P=0.0004; FIG. 12B shows that the MMP19 content in serum is negatively correlated with the estimated glomerular filtration rate eGFR, R=−0.4037, P=0.002.

Chronic kidney disease can be divided into five stages according to the eGFR. CKD I: the eGFR is greater than or equal to 90 ml/min, but there is a history of renal structural changes or kidney injury; CKD II: the eGFR is between 60 ml/min and 89 ml/min; CKD III: the eGFR is between 30 ml/min and 59 ml/min; CKD IV: the eGFR is between 15 ml/min and 29 ml/min; CKD V: eGFR is less than 15 ml/min. As shown in FIG. 13, in this example, the 40 samples from patients with renal puncture biopsy include: 20 CKD I-II serum samples, with the average of MMP19 content in serum being 376.5±29.96 ng/ml; and a total of 20 CKD III-V serum samples, with the average of serum MMP19 level being 468.5±55.96 ng/ml.

This example also collected clinical CKD urine samples, including 25 healthy control examples and a total of 85 cases from patients with renal puncture biopsy. An analysis of the healthy control group and CKD cases showed that the content of MMP19 in the urine of CKD patients increased significantly. The average of MMP19 content in the urine of healthy control group was 0.3896±0.04234 ng/ml; the average of MMP19 content in urine of CKD patients was 2.126±0.4031 ng/ml (FIG. 14A), and the area under the curve of MMP19 content for the diagnosis of CKD was 0.8014 (FIG. 14B).

85 clinical CKD urine samples were staged according to eGFR. As shown in FIG. 15, there were 62 CKD I-II urine samples, with the average of MMP19 content in urine being 1.069±0.2041 ng/ml; a total of 20 CKD III-IV urine samples, with the average of MMP19 content in urine being 3.680±0.9678 ng/ml; and a total of 3 CKD V urine samples, with the average of MMP19 content in urine being 13.6±4.741 ng/ml.

In summary, MMP19 can be used as a biomarker for early diagnosis of acute kidney injury and chronic kidney disease. An existing MMP19 test kit or a self-developed MMP19 test kit was used as an early diagnosis kit for kidney failure. As shown in FIG. 16, the technical route for developing the early diagnosis kit for kidney failure includes the following procedures (taking the MMP19 ELISA kit as an example):
1. preparation of a biological marker MMP19 antibody and standard product, and experimental data detection of the standard product;
2. registration and declaration for the MMP19 ELISA kit;
3. test the produced ELISA kit in parallel with the control reagent to see the difference therebetween, evaluate the stability, specificity, and sensitivity of the ELISA kit, and form an evaluation report; and
4. put the MMP19 ELISA test kit into production, supply the kit to the laboratory or nephrology departments of major tertiary hospitals across the country, set up fixed sales channels, and quickly enter and expand the market.

The above detailed description of the disclosure is intended to enable persons familiar with the art to understand the contents of the disclosure and implement them, instead of limiting the scope of the disclosure. Any equivalent changes or modifications made according to the spirit of the disclosure shall fall within the scope of the disclosure.

1. Use of a reagent for detecting a biomarker in a sample in the preparation of a product for early diagnosis of kidney injury, wherein the biomarker is MMP19.

2. The use according to claim 1, wherein the reagent for detecting a biomarker in a sample comprises a reagent for detecting the expression level and/or activity of the biomarker's mRNA in the sample, and/or, the reagent for detecting a biomarker in a sample comprises a reagent for detecting the expression level and/or activity of the biomarker's protein in the sample.

3. The use according to claim 2, wherein the reagent is one or more of a primer for specifically amplifying the biomarker, a probe for specifically identifying the biomarker, a conjugate that specifically binds to a protein encoded by the biomarker, or a reagent capable of detecting the activity of the biomarker.

4. The use according to claim 3, wherein the conjugate comprises one or more of an antibody, a functional antibody fragment or an antibody conjugate that specifically binds to the protein encoded by the biomarker.

5. The use according to claim 1, wherein a test sample is obtained from a subject, the expression level and/or activity of the biomarker MMP19 in the test sample is compared with that in a normal control, and if the expression level and/or activity of the biomarker MMP19 is increased, the subject is determined to have kidney injury or a tendency to suffer from kidney disease or a recurrence of kidney disease or a poor prognosis of kidney disease.

6. The use according to claim 1, wherein the kidney injury comprises acute kidney injury and/or chronic kidney disease.

7. The use according to claim 1, wherein a test sample is blood and/or urine.

8. The use according to claim 1, wherein the kidney injury is tubulointerstitial injury.

9. A product for early diagnosis of kidney injury, wherein the product comprises a reagent for detecting a biomarker MMP19.

10. The product according to claim 9, wherein the reagent for detecting the biomarker MMP19 is one or more of a primer for specifically amplifying the biomarker MMP19, a probe for specifically identifying the biomarker MMP19, a conjugate that specifically binds to a protein encoded by the biomarker MMP19, or a reagent capable of detecting the activity of the MMP19.

11. The product according to claim 9, wherein the product comprises a test paper, a reagent strip, a kit, a chip, and a high-throughput sequencing platform.

12. The product according to claim 9, wherein the product further comprises a detection component and a result determining component, wherein the detection component is configured to detect the expression level and/or activity of the biomarker MMP19, and the result determining component is configured to determine, based on the expression level and/or activity of the biomarker MMP19 detected by the detection component, whether a subject has kidney injury or is at risk of kidney disease.

13. The product according to claim 12, wherein the detection component comprises a detection device matched with a kit, and the kit comprises the reagent for detecting the biomarker MMP19,
the kit is selected from a qPCR kit, an ELISA kit, an immunoblotting kit, a flow cytometry kit, an immunohistochemistry kit, an immunochromatography kit, and an electrogenerated chemiluminescence kit;
the detection device is selected from a qPCR amplifier, an ELISA device, an immunoblotting device, a flow cytometry device, an immunohistochemistry device, an immunochromatography device, an electrogenerated chemiluminescence device, and an activity test kit device;
the result determining component comprises an input module, an analysis module and an output module;
wherein the input module is configured to input the expression level and/or activity of the biomarker MMP19; the analysis module is configured to analyze, based on the expression level and/or activity of the biomarker MMP19, whether the subject has kidney injury or is at risk of kidney disease; the output module is configured to output an analysis result of the analysis module.

14. A method for early diagnosis of kidney injury, comprising:
a) obtaining a test sample from a subject;
b) detecting the expression level and/or activity of a biomarker MMP19 in the test sample;
c) and comparing a detection result with a normal control, and if the expression level and/or activity of the biomarker MMP19 in the test sample is increased, determining that the subject has kidney injury or a tendency to kidney disease or a recurrence of kidney disease or a poor prognosis of kidney disease.

15. The method according to claim 14, wherein the test sample is blood and/or urine.

16. The method according to claim 14, wherein the kidney injury comprises acute kidney injury and/or chronic kidney disease.

17. The method according to claim 14, wherein the kidney injury is tubulointerstitial injury.

18. The method according to claim 14, wherein the expression level and/or activity of a biomarker MMP19 comprises the expression level and/or activity of the biomarker's mRNA, and/or, the expression level and/or activity of the biomarker's protein.

19. The method according to claim 14, wherein a reagent for detecting the biomarker is one or more of a primer for specifically amplifying the biomarker, a probe for specifically identifying the biomarker, a conjugate that specifically binds to a protein encoded by the biomarker, or a reagent capable of detecting the activity of the biomarker.

20. The method according to claim 19, wherein the conjugate comprises one or more of an antibody, a functional antibody fragment or an antibody conjugate that specifically binds to the protein encoded by the biomarker.
